(19) United States
(12) Patent Application Publication
(10) Pub. No.: US 2006/0137932 A1
(43) Pub. Date: Jun. 29, 2006

(54) Personal Pneumatic Amplification System for an Electric Guitar

(76) Inventor: Richard P. Hatch, Salt Lake City, UT (US)

Correspondence Address: GEOFFREY E. DOBBIN, PATENT ATTORNEY, 4278 SOUTH 6220 WEST, WEST VALLEY CITY, UT, 84128-6501, US

(21) Appl. No.: 10/905,308

(22) Filed: Dec. 27, 2004

(51) Int. Cl. A61B 7/02

(52) U.S. Cl. 181/131

(57) ABSTRACT

The present invention is a pneumatically driven personal amplification system for use with an electric guitar. The invention has three embodiments, each one having a having a listening apparatus connected to an electric guitar through pneumatic tubing. The first embodiment utilizes a plug, disposed on the end of the tubing opposite the listening apparatus and designed to interface with the guitar's output jack. The second embodiment utilizes a detachable diaphragm, localized on the surface of the guitar. The third embodiment is an electric guitar with a permanent diaphragm attached therein and an output port constructed for detachable pneumatic tubing. In all embodiments, volume and control and filters and specialized diaphragm structures may be employed to adjust the sound transmitted to the user.

### FIELD OF THE INVENTION

The present invention relates to the field of musical instruments and more particularly relates to a pneumatically driven personal amplification system for use with an electric guitar.

### BACKGROUND OF THE INVENTION

Electric and electric bass guitars (collectively referred to as “electric guitars”) are a familiar presence in today's music scene. However, due to their construction, they require the addition of an amplification system in order to be heard—even by the player. The addition of the amplifier, however, often creates a noisy disruption while a player is practicing or learning the basics of playing an electric guitar. Players are, therefore in a situation whereby in order to improve they need to practice, but windows of opportunity for practicing are limited. To this end, personal amplification systems have been developed to provide larger windows of opportunity.

Personal amplification systems tend to fall into two categories: pneumatic-driven amplification systems or electronic-driven amplification systems. Electronic systems tend to require a power source, such as a battery, in order to operate. Pneumatic systems, up to this point, have not achieved the simplicity of the electronic amplification systems, which is to say they require special pickup devices in order to work. Often times the pick-up devices are bulky or unwieldy, or require modification to the instrument.

While the aforementioned types of inventions accomplish their individual objectives, they do not disclose a pneumatic personal amplification system that is designed to interface with the instrument without modification or an extensive pick-up system. In this respect, the personal amplification system according to the present invention departs substantially from the usual designs in the prior art. In doing so, this invention provides a pneumatic personal amplification system that offers the ease of a compatible direct connection to either the existing output port or any other location on an electric guitar. This invention also provides a new guitar, with a separate port and diaphragm assembly, which also provides easy connectivity to the listening portion of the amplification system.

### SUMMARY OF THE INVENTION

In view of the foregoing disadvantages inherent in the known types of personal amplification systems, this invention provides an improved amplification system featuring pneumatic sound transfer and easier connection to the instrument. As such, the present invention's general purpose is to provide a new and improved amplification system that will interface directly with the instrument, ideally on the surface of the instrument, and pneumatically transmit signal to the user.

To accomplish this purpose, the amplification system is comprised of either an interface jack or diaphragm connected to pneumatic tubing. The tubing is then further connected to a listening apparatus that directs the sound waves to the user's ears. The jack is inserted into the instrument's output port and sound waves are then transmitted through the column of closed air to the user's ears. Alternate interface options with the instrument exist. These options include directly attaching a diaphragm to the instrument's exterior or even installing a pick-up diaphragm inside the instrument and having an outlet port for attachment of pneumatic tubing.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a plan view of the present invention connected to an electric guitar.

FIG. 2 is a close-up plan view of the pickup jack, as described, and an output jack's interior clipping mechanism.

FIG. 3 is a partial cross-section of the guitar of FIG. 1, detailing the interior circuitry and resonance chamber inside an electric guitar.

FIG. 4 is a blown-apart view of the present invention.

FIG. 5 is a blown-apart view of an alternative embodiment of the present invention.

FIG. 6 is a plan view of the invention embodiment of FIG. 5 in use with an electric guitar.

FIG. 7 is a close-up view of the embodiment in claim 6, detailing the connection of the pickup diaphragm and the pneumatic tubing.

FIG. 8 is a side-plan view of the modified pick-up diaphragm structure.

FIG. 9 is a blown apart view of the modified pick-up diaphragm.

FIG. 10 is a plan view of a standard diaphragm disc.

FIG. 111 is a plan view of a modified, low volume diaphragm disc.

### DETAILED DESCRIPTION OF PREFERRED EMBODIMENTS

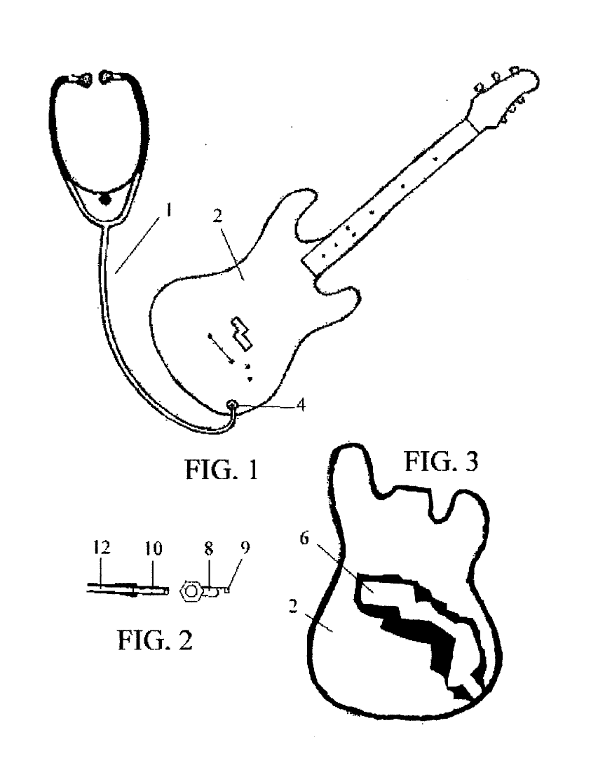

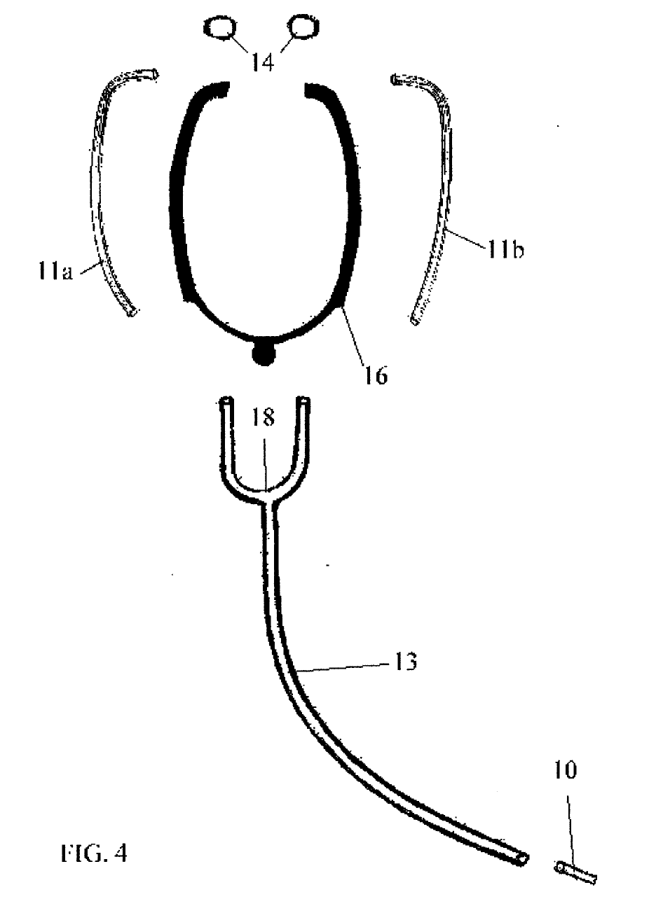

With reference now to the drawings, the preferred embodiment of the personal amplification system is herein described. Referring to FIG. 1, the amplification system 1 is attached to guitar 2 at output port 4. Hollow pick-up jack 10, shown in FIG. 4, pneumatic tubing 12 and hollow earpieces 14, defines an air column. All of these parts of the invention being in fluidic communication with each other and resonance chamber 6, shown in FIG. 3 through port 4. Headset 16 is provided for which to hold pneumatic tubing 12 and earpieces 14 in a manner more comfortable for the user. The actual shape and structure of headset 16 is irrelevant, so long as it is capable of receiving the pneumatic tubing 12 and holding it in a manner that directs earpieces 14 towards the user's eardrums. Additional headsets may be attached to allow for practice sessions and lessons, though further division of tubing will affect end volume.

Pick-up jack 10 interfaces with port 4 in its contained clip 8, shown in FIG. 2. The dip 8 is actually manufactured as a part of the circuitry system of the electric guitar 2 and must be treated accordingly. As such, the preferred material from which jack 10 is to be manufactured is plastic. It must also be of sufficient length to securely interface with clip 8, while not being so long to damage it, specifically by disengaging the back contact 9 of the clip 8.

In use, the jack 10, when inserted into port 4 is in fluidic contact with chamber 6. When the instrument is played, sound vibrations are generated by the strings and are transmitted to the resonance chamber. Normally, these vibrations are picked up and transmitted through electric circuitry to an amplifier; however, with the present invention, the sound waves travel into the pick-up jack 10 and through the column of air defined by pneumatic tubing 12. The waves exit the column through the earpieces 14. The waves are very faint, requiring that they be directed almost directly into the user's ear canal; but, through the use of this invention 1, the user may play the instrument without bothering or distracting those around the user, as the electric guitar is almost silent without an amplifier system. Ideally, pneumatic tubing 12 should be split 18 so as to direct sound to both of a user's ears, but the split is unnecessary to the essence of the invention. Likewise, pneumatic tubing 12 may be a series of tubes 11a, 11b, and 13, interconnected, as shown in FIG. 4.

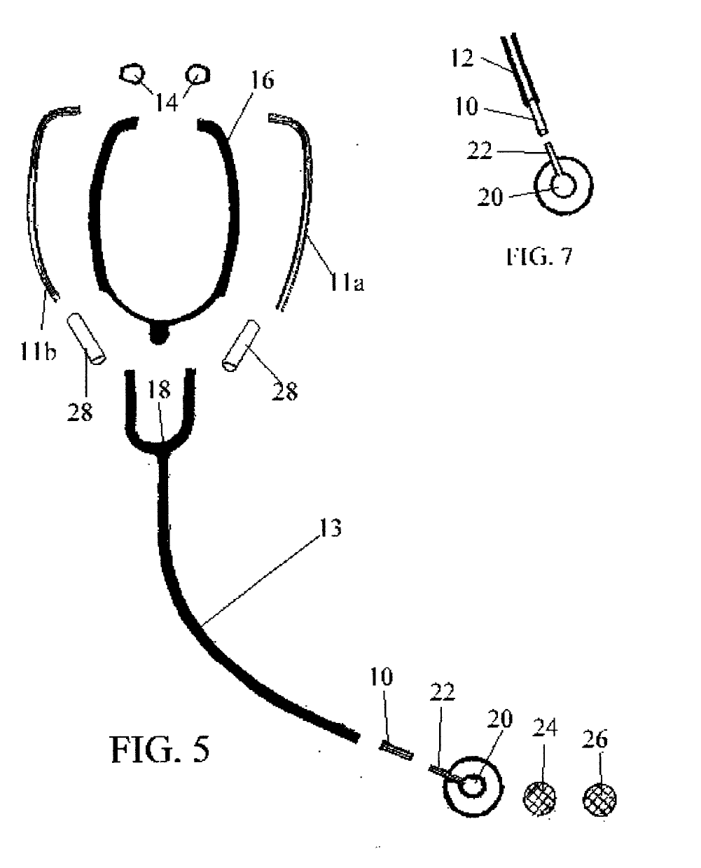

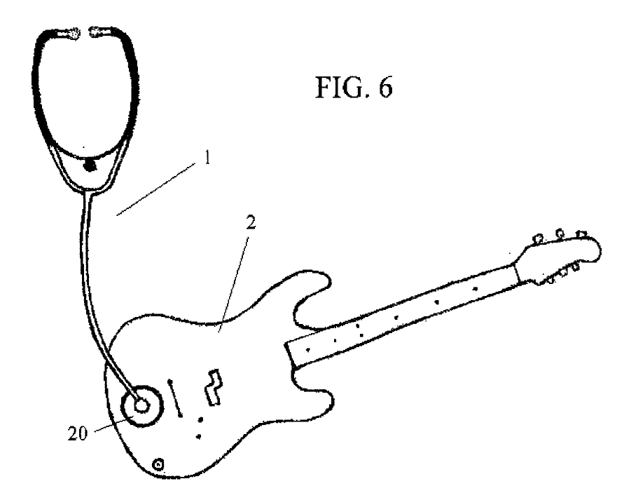

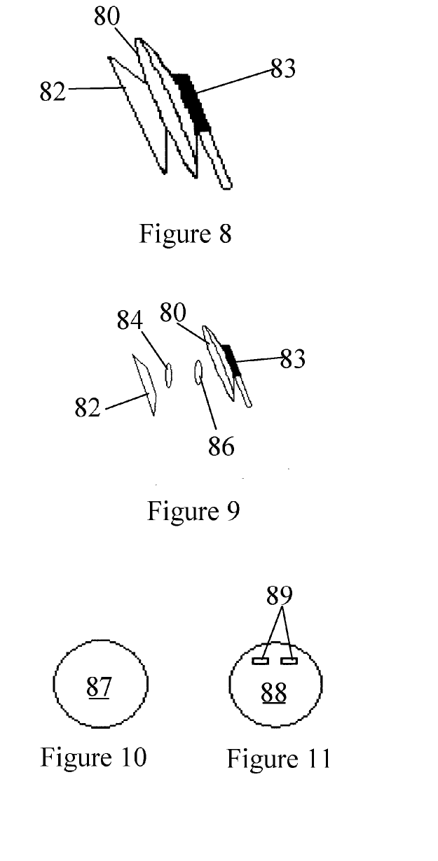

In an alternative embodiment, shown in FIGS. 5 and 6, a diaphragm structure 20 may be added. This structure may be external or internal of the instrument. If it is external, as shown in FIG. 5. The ideal means of connecting the diaphragm to the instrument 2 is shown in FIGS. 8 and 9. Pick-up diaphragm 80 is connected to suction cup 82 with hook and loop fastening discs 84, 86. Any attachment method is possible, including epoxies, resins, glues and other adhesive and bonding techniques, however permanent means of attaching the suction cup 82 to diaphragm 80 may result in the diaphragm 80 being removed from the remainder of the structure 83 when the invention is removed from the surface of a guitar. Hook and loop fastener pads, 24, 26 shown in FIG. 5, may be provided for a semi-permanent affixation. Otherwise, permanent-bonding techniques, such as glues, resins and epoxies, may be employed. The diaphragm structure 20 may also be installed within resonance chamber 6, with outlet port 22 extending through the walls of the instrument 2. In either embodiment, pickup jack 10 interfaces with outlet port 22 to create the necessary air column FIG. 7.

In all embodiments, a volume control or filter structure 28 may be added so as to adjust the volume or sound quality and intensity of the transmitted sound waves. The structure may be an additional filter component 28 or an altered diaphragm structure, as shown in FIGS. 10 and 11. FIG. 10 depicts a normal diaphragm 87 while FIG. 11 depicts a low-volume diaphragm 88 with volume control holes 89 which lessen the intensity of sound waves transmitted to the user's ear canal. Filter components 28 may be constructed to increase or decrease volume or otherwise alter tone quality of the sound waves. Methods of construction of such filters include closable valves on the tube body, and insertable materials to alter the sound waves.

Although the present invention has been described with reference to preferred embodiments, numerous modifications and variations can be made and still the result will come within the scope of the invention. No limitation with respect to the specific embodiments disclosed herein is intended or should be inferred.

What is claimed is:

1. A personal pneumatic amplification system for an electric guitar, the system comprising:
a hollow insert, insertable within the guitar body, defining a column of air;
pneumatic tubing, one end being disposed upon the insert and in pneumatic connection with a column of air defined by the insert, the other end being a listening end; and
at least one earpiece structure, capable of directing sound waves;
wherein, a listening end of the pneumatic tubing passes into the at least one earpiece and sound is directed towards the user's ear canal.

2. The system of claim 1, further comprising a dividing structure within the pneumatic tubing, forming a plurality of listening ends opposite the end disposed upon the insert.

3. The system of claim 2, further comprising a plurality of earpieces, matched in number to the plurality of listening ends, a single earpiece being mounted upon a single listening end.

4. The system of claim 3, further comprising a filter component.

5. The system of claim 3, the plurality of listening ends being directed through a plurality of headsets.

6. The system of claim 5, further comprising a filter component.

7. The system of claim 1, further comprising a filter component.

8. A personal pneumatic amplification system for an electric guitar, the system comprising: a pickup diaphragm structure; pneumatic tubing, one end being disposed upon the diaphragm; and at least one earpiece structure, capable of directing sound waves; wherein a listening end of the pneumatic tubing passes into the at least one earpiece and sound is directed towards the user's ear canal.

9. The system of claim 8, further comprising a dividing structure within the pneumatic tubing, forming a plurality of listening ends opposite the end disposed upon the insert.

10. The system of claim 9, further comprising a plurality of earpieces, matched in number to the plurality of listening ends, a single earpiece being mounted upon a single listening end.

11. The system of claim 10, further comprising a pick-up diaphragm having at least one perforation.

12. The system of claim 9, the plurality of listening ends being directed through a plurality of headsets.

13. The system of claim 12, the diaphragm structure further comprising: a pick-up diaphragm; a suction cup having a flat attachment surface; attachment means linking the diaphragm and suction cup.

14. The system of claim 13, further comprising a pick-up diaphragm disc having at least one perforation.

15. The system of claim 12, further comprising a pick-up diaphragm disc having at least one perforation.

16. The system of claim 8, the diaphragm structure further comprising: a pick-up diaphragm; a suction cup having a flat attachment surface; attachment means linking the diaphragm and suction cup.

17. The system of claim 16, further comprising a pick-up diaphragm disc having at least one perforation.

18. The system of claim 8, the diaphragm being permanently mounted upon an electric guitar and having an outlet port whereby the pneumatic tubing may be mounted and be pneumatically connected to the diaphragm structure.

19. The system of claim 18, further comprising a dividing structure within the pneumatic tubing, forming a plurality of listening ends opposite the end disposed upon the insert.

20. The system of claim 19, further comprising a plurality of earpieces, matched in number to the plurality of listening ends, a single earpiece being mounted upon a single listening end.

21. The system of claim 20, further comprising a filter component.

22. The system of claim 19, the plurality of listening ends being directed through a plurality of headsets.

23. The system of claim 22, further comprising a filter component.

24. The system of claim 18, further comprising a filter component.
